(19) United States
(12) Patent Application Publication
(10) Pub. No.: US 2005/0056284 A1
(43) Pub. Date: Mar. 17, 2005

(54) Electronic breathing system

(76) Inventors: Fred Rascoe, Lawrenceville, GA (US); Chai Xiong, Monroe, GA (US); Barry Martin, Cleveland, TN (US); Bob Baron, Covington, GA (US)

Correspondence Address: Walter A. Rodgers;Rodgers & Rodgers, 880 N. Island Drive, Atlanta, GA, 30327, US

(21) Appl. No.: 10/661,838

(22) Filed: Sep. 15, 2003

(51) Int. Cl. A61M 16/00

(52) U.S. Cl. 128/204.26; 128/205.25; 128/204.18; 128/204.21; 128/204.22

(57) ABSTRACT

An electronic breathing system comprising a facemask for receiving compressed air, a first stage regulator to reduce the air pressure, a demand valve to control the flow of air to the facemask, an exhalation valve to control the flow of air from the facemask, a pressure transducer to detect changes in pressure in the facemask, and a microprocessor to control the opening and closing of the valves.

### BACKGROUND OF THE INVENTION

Second stage regulators or demand valves for use in connection with self-contained breathing equipment have historically been totally mechanically operated. Through the use of elastomeric diaphragms, which flex at low differential pressures, a unit responds to minute changes in air pressure within the facemask. The premise of the design is to create a positive pressure using breathing air (above 0 inch of water gage) inside the mask. NIOSH mandates that the pressure be between 0 inch of water and 3.5 inches of water throughout the entire breathing cycle.

Typically, the inhalation pressure will be between 0 and 0.5 inch and exhalation between 1.8 and 2.5 inches of water which require effort and exertion on the part of the wearer to breathe in this environment. Typical exhalation valves operate on positive spring pressure against an elastomeric flap. When the exhalation pressure reaches a predetermined limit (typically 2.5 inch of water), the force against the spring and flap assembly causes the flap to open and allows air to escape.

### SUMMARY OF THE INVENTION

An electronic breathing system wherein compressed air is supplied to an electronic demand valve which opens in response to a drop in facemask pressure and wherein an increase in pressure in the facemask causes an electronic exhalation valve to open and allow air to escape to atmosphere. A pressure transducer detects changes in facemask pressure and signals a microprocessor to initiate operation of the inhalation and exhalation valves.

### BRIEF DESCRIPTION OF THE SEVERAL VIEWS OF THE DRAWINGS

In the drawings,

FIG. 1 is a schematic diagram of the electronic breathing system according to this invention; and

FIG. 2 is a schematic representation depicting the breath cycle when the breathing apparatus is in use.

### DETAILED DESCRIPTION OF THE INVENTION

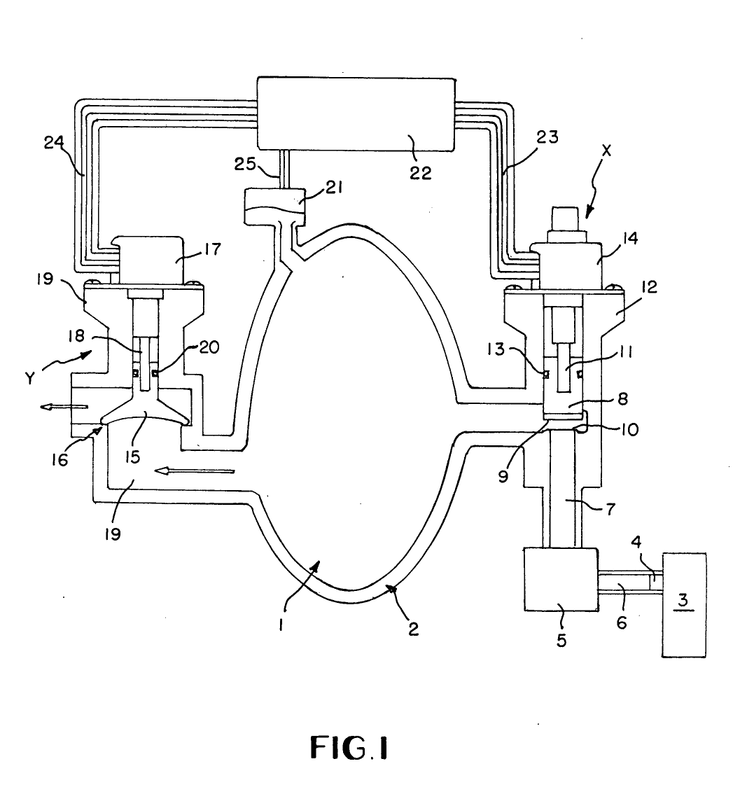

In the drawings and with particular reference to FIG. 1, the numeral 1 designates the apparatus facemask which covers the eyes, nose and mouth of the user, and includes visor 2, as is well known. Cylinder 3 and associated valve assembly 4 is the storage component for the air supply. Air is stored in a compressed state in cylinder 3 and, through the operation of valve 4, flows to first stage regulator 5. Operating pressures from cylinder 3 range up to 4500 psi.

First stage regulator 5 receives the compressed air by means of airline 6 and operates to reduce the pressure from cylinder 3 to a lower pressure, typically from 50 psi to 120 psi. First stage regulator 5 then delivers air under reduced pressure to the electronic demand valve via airline 7.

The electronic demand valve is generally designated by the letter X and receives air from first stage regulator 5 whereby piston 8 moves in a linear fashion so as to control the volume of air entering facemask 1. Piston 8 normally closes the valve whereby piston seat pad 9 makes contact with valve seat 10 which stops the air from flowing. When flow is required, piston 8 moves upwardly away from seat 10. Piston 8 is housed in valve body 12 and sealed by means of O-ring seal 13 and is attached to shaft 11 which is moved linearly by means of motor 14. The amount of air required and delivered is in direct proportion to the gap between seat pad 9 and valve seat 10. More air requires a larger gap and less air requires a smaller gap.

The electronic exhalation valve is shown in FIG. 1 and is identified generally by the letter Y and functions to control the air flow to outside ambient air from facemask 1. Piston 15 moves linearly from valve seat 16 in similar fashion to electronic demand valve X by means of motor 17 and associated shaft 18. Typically, piston 15 rests on valve seat 16 so as to close the valve and thus allow no air to escape. Also piston 15 is housed in valve body 19 and is sealed by means of O-ring seal 20.

As the pressure inside facemask 1 increases due to volume decrease, i.e., expelling air from the user's lungs, valve 15 moves away from seat 16 to allow air to escape from the facemask. Pressure transducer 21 monitors the pressure inside facemask 1 and, as the pressure changes, pressure transducer 21 detects the change and transmits this information to microprocessor 22 for processing. Pressure transducer 21 typically operates in the range from 0 inch of water pressure to 4 inches of water pressure. Finally, demand valve X and exhalation valve Y are connected, respectively, to microprocessor 22 by means of electronic connectors 23 and 24 and pressure transducer 21 is connected to microprocessor 22 by means of electronic connection 25.

Microprocessor 22 is the electronic and microprocessing element of the system. Microprocessor 22 controls all of the valves in the system whereby it receives data from pressure transducer 21 and determines if air needs to be supplied to facemask 1 by means of electronic demand valve X or if air needs to be released from facemask 1 via electronic exhalation valve Y.

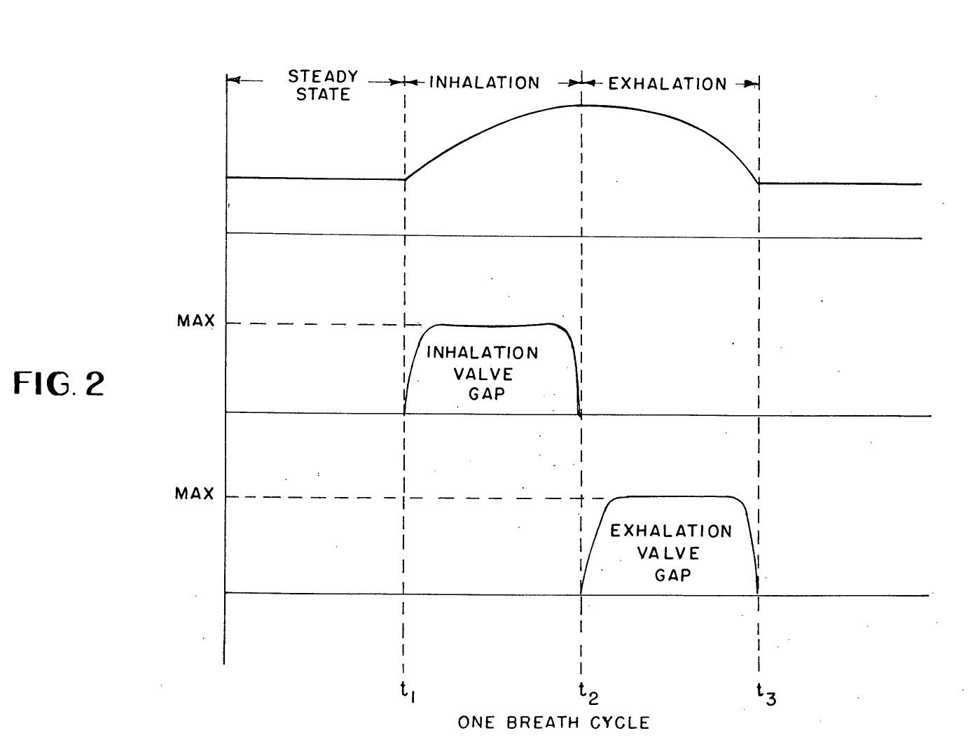

A typical breathing cycle is shown in FIG. 2 in which initially in the steady state there is no breathing and the gaps for the electronic exhalation valve and electronic demand valve are zero. The pressure transducer will detect the steady state pressure which is typically around one inch of the water static.

At time t1, the user starts inhalation which causes the control volume to increase which in turn causes a momentary drop in pressure inside facemask 1. Pressure transducer 21 detects a momentary drop in pressure and feeds the data to microprocessor 22. Microprocessor 22 then sends a command to electronic demand valve motor 14 to open piston 8 and allow air to enter the facemask to bring the pressure back up to the static level, typically to 1.0 to 1.2 inches of water. As inhalation increases, microprocessor 22 monitors the pressure level in the facemask to maintain it at a typical static pressure until time t2 when the user stops inhalation. At this time, pressure stabilizes to a static level and electronic demand valve X shuts off.

At time t2, when the user starts exhalation, the pressure inside facemask 1 begins to increase due to the volume change. Pressure transducer 21 detects the pressure increase and sends the data to microprocessor 22 which then sends commands to motor 17 to open piston 15 and allow air to escape thus lowering the pressure to a static level which continues until time t3 at which time exhalation ends.

1. An electronic breathing system comprising a source of compressed air, a facemask for receiving air from said source, a demand valve responsive to a decrease in pressure in said facemask to regulate the flow of air to said facemask, a first stage regulator disposed between said source of compressed air and said demand valve and operative to reduce the pressure of said compressed air to approximately 50 to 120 psi, an exhalation valve responsive to an increase in pressure in said facemask to allow air to escape from said facemask, a pressure transducer to monitor pressure in said facemask, and a microprocessor to control the operation of said demand valve and said exalation valve.

2. A system according to claim 1 wherein said demand valve comprises a piston linearly moveable by means of a motor.

3. A system according to claim 2 wherein said piston comprises a seat pad which cooperates with a valve seat to control the flow of air to said facemask.

4. A system according to claim 1 wherein said exhalation valve comprises a piston linearly moveable by means of a motor.

5. A system according to claim 4 wherein said piston is housed in a valve body.

6. A system according to claim 4 wherein said piston cooperates with a valve seat to control the flow of air from said facemask.
